(19) United States
(12) Patent Application Publication
(10) Pub. No.: US 2021/0171468 A1
(43) Pub. Date: Jun. 10, 2021

(54) PROCESS FOR THE MANUFACTURE OF PYRAZOLE CARBOXYLIC DERIVATIVES AND PRECURSORS THEREOF

(71) Applicant: SOLVAY SA, Brussels (BE)

(72) Inventors: Janis JAUNZEMS, Hannover (DE); Alexander SCHULZ, Hannover (DE); Uta CLAASSEN, Hohenhameln (DE)

(21) Appl. No.: 16/771,043

(22) PCT Filed: Dec. 20, 2018
(86) PCT No.: PCT/EP2018/086258

§ 371 Date: Jun. 9, 2020

(30) Foreign Application Priority Data
Dec. 22, 2017 (EP) 17210019.0

(51) Int. Cl. C07D 231/14; C07C 221/00; C07C 249/16; C07D 231/12

(52) U.S. Cl. CPC C07D 231/14; C07C 221/00; C07C 225/14; C07D 231/12; C07C 249/16

(57) ABSTRACT

The present invention concerns processes for the manufacture of pyrazole carboxylic derivatives and precursors thereof.

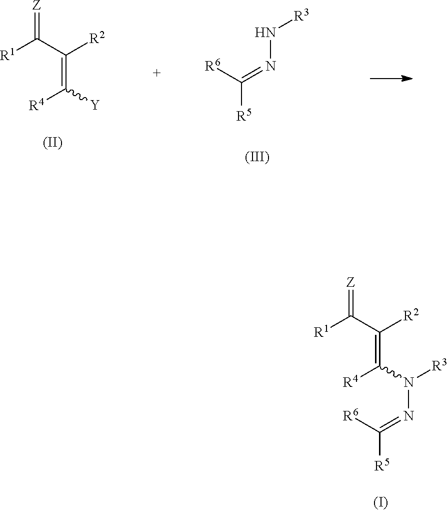

This application claims priority to European application No. 17210019.0, the whole content of this application being incorporated herein by reference for all purposes.

The present invention concerns processes for the manufacture of pyrazole carboxylic derivatives and precursors thereof.

Substituted pyrazoles carboxylic acid derivatives, in particular 3-halomethylpyrazole-4-yl carboxylic derivatives, are valuable intermediates in the synthesis of agrochemical and pharmaceutical active ingredients. Agrochemical active ingredients which contain such pyrazole building blocks are, for example, 2′-[1,1′-bicycloprop-2-yl]-3-(difluoromethyl)-1-methylpyrazole-4-carboxanilide (Sedaxane), as described, for example, in WO2006015866, 3-(difluoromethyl)-1-methyl-N-[2-(3′,4′,5′-trifluorophenyl)phenyl]pyrazole-4-carboxamide (Fluxapyroxad), as described, for example, in WO2006087343, N-(3′,4′-Dichloro-5-fluorobiphenyl-2-yl)-3-(difluoromethyl)-1-methylpyrazole-4-carboxamide (Bixafen), as described, for example, in WO2003070705, 3-(Difluoromethyl)-1-methyl-N-[1,2,3,4-tetrahydro-9-(1-methylethyl)-1,4-methanonaphthalen-5-yl]-1H-pyrazole-4-carboxamide (Isopyrazam), as described, for example, in WO2004035589, (RS)—N-[9-(Dichloromethylen)-1,2,3,4-tetrahydro-1,4-methanonaphthalin-5-yl]-3-(difluoromethyl)-1-methyl-1H-pyrazole-4-carboxamide (Benzovindiflupyr), as described, for example, in WO07048556. Generally, 3-halomethylpyrazole-4-yl carboxylic acids, often obtained by hydrolysis of their esters, are converted into the carboxamides, for example after conversion into the 3-halomethylpyrazole-4-yl carboxylic acid halide. Other conversions, wherein the carboxamide is generated directly from the ester or acid, have also been described, such as in WO2012055864 and WO 2007/031323. All foregoing cited patent applications are hereby incorporated for all purposes.

In the manufacture of pyrazoles intermediates, often unsaturated acyl derivatives are cyclized with hydrazine or hydrazine derived compounds. The optimization of regioselectivity in such a cyclization reaction has been reported to be achieved by using hydrazine derivatives such as hydrazones instead of hydrazines. EP2247577A discloses that in order to induce satisfactory reaction between the unsaturated acyl derivatives or acrylic acid derivatives which are imino-compounds with the hydrazone compound, a catalyst is required, which is an acidic catalyst such as KHSO₄, HCl or H₂SO₄. Such acids can also promote disintegration of the hydrazone compound, releasing the hydrazine, which can then react with reduced or absent regioselectivity with the unsaturated acyl derivatives to form the pyrazoles. Also, the addition of such an acid into the reaction mixture adds to the complexity of the mixture, workup procedure and waste management. Overall, the addition of such acidic catalysts can be disadvantageous.

Surprisingly, has now been found that the reaction of hydrazone compounds with unsaturated acyl derivatives can be enhanced in the presence of an amine salt. The amine salt does not induce decomposition of hydrazone compounds, which preserves the ability of the reaction between unsaturated acyl derivative and hydrazone to react in a regioselective manner. Moreover, the amine salt can be present in the reaction mixture from a step prior to the reaction of the acyl derivative with the hydrazone compound, for example in the step of manufacturing the unsaturated acyl derivative. Thus, the co-product of a step prior to the reaction between the acyl derivative and the hydrazone, rather than being discarded, can act as auxiliary or catalyst of a subsequent step. Manufacture of downstream products, such as pyrazoles compounds, can thus be achieved with higher yields, selectivity, less additional substances employed in the reaction sequence, hence less by-products, waste and workup handling.

The invention thus concerns a process for manufacturing a compound according to formula (I), which comprises the reaction a compound of formula (II) and a compound of formula (III)

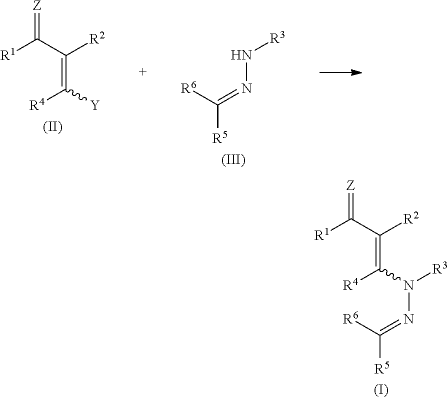

wherein Z, Y and R¹ to R⁶ are defined as in the description, wherein in the reaction of the compound of formula (I) and (II), an amine salt is present.

The invention further concerns a process for the manufacture of a compound of formula (IV),

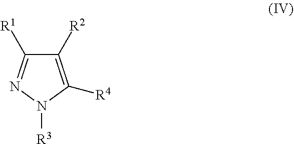

which comprises the process for the manufacture of a compound of formula (I), and a process for the manufacture of a compound of formula (V), which comprises the process for the manufacture of a compound of formula (IV).

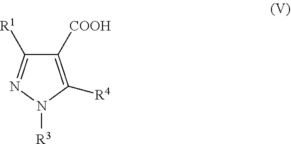

Another object of the present invention is a process for the manufacture of a compound of formula (VI)

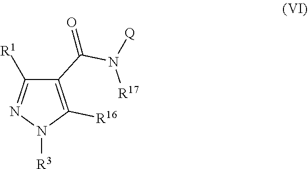

wherein R¹⁷ and Q are as defined in the subsequent description, which comprises at least one of the processes for the manufacture of a compound of formula (IV) and/or (V).

In the present invention, designations in singular are in intended to include the plural; for example, “a solvent” is intended to denote also “more than one solvent” or “a plurality of solvents”.

All aspects and embodiments of the present invention are combinable.

In the context of the present invention, the term “comprising” is intended to include the meaning of “consisting of”.

When a double bond is depicted in a particular E/Z geometry, this is intended to also denote the other geometric form as well as mixtures thereof.

The invention concerns in a first aspect a process for manufacturing a compound according to formula (I), which comprises the reaction a compound of formula (II) and a compound of formula (III)

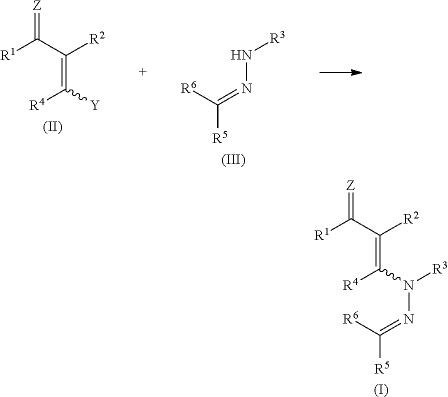

wherein Z is selected from O, S and N⁺R⁷R⁸, wherein R⁷ and R⁸ are independently selected from the group consisting of C₁-C₁₂-alkyl, C₃-C₁₀-cycloalkyl, aryl, heteroaryl and aralkyl groups, each of which is optionally substituted, or wherein R⁷ and R⁸ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members,

wherein R¹ is selected from the group consisting of optionally substituted C₁ to C₄ alkyl groups,

wherein R² is selected from the group consisting of C(O)OR⁹, CN, C(O)R¹⁰ and C(O)NR¹¹R¹², wherein R⁹, R¹⁰, R¹¹ and R¹² each independently are selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted, or wherein R¹¹ and R¹² together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members,

wherein Y is selected of OR¹³, NR¹⁴R¹⁵ and SR¹⁶, wherein R¹³, R¹⁴, R¹⁵ and R¹⁶ each independently are selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted, or wherein R¹⁴ and R¹⁵ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members,

wherein R³ is selected from the group consisting of H, C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted,

wherein R⁵ and R⁶ each independently are selected from the group consisting of H, C₁-C₁₂-alkyl, C₂-C₆ alkenyl, C₃-C₁₀-cycloalkyl, C₂₋₁₂ alkynyl, aryl, heteroaryl or aralkyl group, each of which is optionally substituted, wherein at least one of R⁵ and R⁶ is different from H,

wherein R⁴ is selected from the group consisting of H, X′, COOR′, OR′, SR′, C(O)NR′₂, wherein the groups R′ are selected independently in C(O)NR′₂ where R′ is selected from the group consisting of hydrogen, C₁-C₁₂-alkyl, CN, C₁-C₁₂-alkyl, C₂-C₆ alkenyl, aryl, cycloalkyl, aralkyl and heteroaryl, each of which is optionally substituted, and wherein X′ is a halogen atom

wherein in the reaction of the compound of formula (I) and (II), an amine salt is present.

In the context of the present invention, the term “C₁-C₁₂-alkyl groups” is intended to denote straight or branched alkyl groups having one to twelve carbon atoms. The group comprises, for example, n-nonyl and its isomers, n-decyl and its isomers, n-undecyl and its isomers and n-dodecyl and its isomers, methyl, ethyl, n-propyl, isopropyl, n-, iso-, sec- and t-butyl, n-pentyl and its isomers, n-hexyl and its isomers, 1,3-dimethylbutyl, 3,3-dimethylbutyl, n-heptyl and its isomers and n-octyl and its isomers. Often, the C₁ to C₄ alkyl groups are the most preferred groups of the C₁-C₁₂ alkyl group. The term “C₁-C₄-alkyl group” is intended to denote straight or branched alkyl groups having one to four carbon atoms. This group comprises methyl, ethyl, n-propyl, isopropyl, n-, iso-, sec- and t-butyl. Where indicated, an alkyl group can optionally be substituted by one or more substituents of the group S*, wherein S* consists of R′, —X′, —OR′, —SR′, —NR′₂, —SiR′₃, —COOR′, —(C═O)R′, —CN and —CONR′₂, wherein R′ is selected independently from the group consisting of hydrogen and C₁-C₁₂-alkyl groups and X′ is selected from the group consisting of F, Cl, Br, or I.

The term “C₂-C₆ alkenyl” intends to denote a group comprising a carbon chain and at least one double bond. Alkenyl group are, for example, ethenyl, propenyl, butenyl, pentenyl or hexenyl. Where indicated, a C₂-C₆ alkenyl group can optionally be substituted by one or more substituents of the group S* as defined above.

The term “C₃-C₁₀-cycloalkyl” intends to denote mono-, bi- or tricyclic hydrocarbon groups comprising 3 to 10 carbon atoms, in particular 3 to 6 carbon atoms. Examples of monocyclic groups include cyclopropyl, cyclobutyl, cyclopentyl, cyclohexyl, cycloheptyl or cyclooctyl. Examples of bicyclic groups include bicyclo[2.2.1]heptyl, bicyclo[3.1.1]heptyl, bicyclo[2.2.2]octyl and bicyclo[3.2.1]octyl. Examples of tricyclic groups are adamantyl and homoadamantyl. Where indicated, a C₃-C₁₀-cycloalkyl group can optionally be substituted by one or more substituents of the group S* as defined above.

In the context of the present invention, C₂₋₁₂ alkynyl groups are, unless defined otherwise, straight-chain, branched or cyclic hydrocarbon groups which contain at least one double unsaturation (triple bond) and may optionally have one, two or more single or double unsaturations or one, two or more heteroatoms selected from the group consisting of O, N, P and S. Where indicated, a C₂₋₁₂-alkynyl group can optionally be substituted by one or more substituents of the group S* as defined above. The definition C₂₋₁₂-alkynyl comprises the largest range defined herein for an alkynyl group. Specifically, this definition comprises, for example, the meanings ethynyl (acetylenyl); prop-1-inyl and prop-2-inyl.

The term “aryl group” intends to denote C₅-C₁₈ monocyclic and polycyclic aromatic hydrocarbons with 5 to 18 carbon atoms in the cyclic system. Specifically, this definition comprises, for example, the meanings cyclopentadienyl, phenyl, cycloheptatrienyl, cyclooctatetraenyl, naphthyl and anthracenyl. Generally, an aryl group can optionally be substituted by one or more substituents of the group S* as defined above.

The term “heteroaryl group” intends to denote C₅-C₁₈ monocyclic and polycyclic aromatic hydrocarbons with 5 to 18 carbon atoms in the cyclic system, wherein one or more methine (—C═) and/or vinylene (—CH═CH—) groups are replaced by trivalent or divalent heteroatoms, in particular nitrogen, oxygen and/or sulphur, respectively, in such a way as to maintain the continuous π-electron system characteristic of aromatic systems. Specifically, this definition comprises, for example, the meanings 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl. Generally, a heteroaryl group can optionally be substituted by one or more substituents of the group S* as defined above.

The term “aralkyl” intends to denote alkyl groups which are substituted by aryl groups, which have a C₁-C₈-alkylene chain and which may be substituted in the aryl skeleton or the alkylene chain by one or more heteroatoms selected from the group consisting of O, N, P and S.

The optionally substituted 5- to 10-membered heterocyclic radical which, in addition to the nitrogen atom, may contain a further 1, 2 or 3 heteroatoms selected from the group consisting of O, N and S as ring members, which can be formed when Y═NR¹⁴R¹⁵ and R¹⁴ and R¹⁵, or R¹¹ and R¹² and the nitrogen atom to which the two radicals are attached form such optionally substituted 5- to 10-membered heterocyclic radical, can be, for example, a pyrrolidin radical, a piperidine radical, a hexamethylenimine radical, a morpholine radical or a thiomorpholine radical.

The term “amine salt” to be present in the reaction of the compound of formula (I) with the compound of formula (II) intends to denote the presence of an amine salt which is different from the compound of formula (I) and/or (II). In one aspect, the amine salt is present before the reaction of the compound (I) and (II), for example in a mixture with the compound of formula (I). Such a mixture can be obtained, for example, by a prior reaction for the manufacture of compound (I), where the amine salt is formed as a co-product. In another aspect, the amine salt is added to the reaction mixture before addition of the compound of formula (I) and/or (II), simultaneously with one of the compounds of formula (I) or (II), or after (I) and (II) are added to the reaction mixture. Generally, catalytic amounts of the amine salt can be sufficient to enhance the reaction between the compound of formula (I) and (II). In another aspect, the amine salt preferably is present in an amount of from 0.8 to 1.3 equivalents, based on the amount of formula (I). In one aspect, the anion of the amine salt is a halide salt with a halide anion, such as F⁻, Cl⁻, Br⁻ or I⁻, wherein F⁻, Cl⁻ and Br⁻ are preferred, and Cl⁻ is most preferred. The amine salt can derive from ammonia, from primary, secondary or tertiary amines, wherein it is most preferred that the amine salt is derived from tertiary amines. Suitable amines are primary, secondary or tertiary aliphatic, cycloaliphatic or aromatic amines, or diamines of up to about 12 carbons. According to a preferred aspect, tertiary aromatic amines are particularly preferred. Examples of suitable amines include ammonia, trimethylamine, trietylamine, dimethylamine, dicyclohexylamine, ethylenediamine, tetramethylethylenediamine, piperidine, pyridine, 2,6-lutidine, 2-methylpyridine, 3-methylpyridine, 4-methylpyridine and dimethylaminopyridine, wherein 2,6-lutidine, 2-methylpyridine, 3-methylpyridine, 4-methylpyridine and pyridine are preferred, and pyridine is most preferred. The amine salts are preferablyhalide salts, for example selected from triethylamine hydrochloride, pyridinium hydrofluoride and pyridinium hydrochloride. The most preferred amine salt is pyridinium hydrochloride. In one aspect, more than one amine salt is present in the reaction of the compound of formula (I) with the compound of formula (II).

According to the present invention, R¹ is selected from the group consisting of optionally substituted C₁ to C₄ alkyl groups. In a preferred aspect, R¹ is selected from the group consisting of C₁ to C₄ alkyl groups, wherein the alkyl group is substituted by at least one halogen atom. The at least one halogen atom is preferably selected from the group consisting of F, Cl, Br and I, wherein F and Cl are preferred. R¹ can be, for example, selected from the group consisting of CF₃, CHF₂, CH₂F, CCl₃, CHCl₂, CH₂Cl, CBr₃, CBr₂H, CBrH₂, CI₃, CI₂H, CBr₂Cl, CCl₂Br, C₂F₅, C₂Br₅ and C₂Cl₅. More preferably, R¹ is selected from the group consisting of CF₃, CHF₂, CCl₃, CHCl₂ and CH₂Cl, wherein CF₃ and CF₂H are preferred, and CHF₂ is most preferred.

In its broadest definition according to the present invention, R² is selected from the group consisting of C(O)OR⁹, CN, C(O)R¹⁰ and C(O)NR¹¹R¹², wherein R⁹, R¹⁰, R¹¹ and R¹² each independently are selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted with at least one of the substituents of the group S* as defined above, or wherein R¹¹ and R¹² together with the nitrogen atom to which they are bound form a substituted 5- to 10-membered heterocyclic radical, which is optionally substituted with at least one of the substituents of the group S* as defined above, which, in addition to the nitrogen atom, may contain a further 1, 2 or 3 heteroatoms selected from the group consisting of O, N and S as ring members. R⁹ preferably is a C₁ to C₄ alkyl group, wherein methyl and ethyl are preferred. R¹¹ and R¹² preferably independently are selected from the group consisting of C₁ to C₄ alkyl groups, wherein methyl and ethyl are preferred. In one aspect, it is particularly preferred that R² is C(O)R¹⁰. R¹⁰ often is selected from the group consisting of C₁ to C₄ alkyl groups which is optionally substituted with at least one of the substituents of the group S* as defined above. In one aspect, R¹⁰ preferably is an unsubstituted methyl group. In another aspect, R¹⁰ is selected from the group consisting of C₁ to C₄ alkyl groups, wherein the alkyl group is substituted by at least one halogen atom. R¹⁰ can be selected, for example, from the group consisting of CF₃, CHF₂, CH₂F, CCl₃, CHCl₂, CH₂Cl, CBr₃, CBr₂H, CBrH₂, CI₃, CI₂H, CBr₂Cl, CCl₂Br, C₂F₅, C₂Br₅ and C₂Cl₅, wherein CF₃, CCl₃, CBr₃, CI₃, C₂Br₅ and C₂Cl₅ are preferred, and CCl₃ is most preferred.

According to the present invention, Z is selected from O, S and N⁺R⁷R⁸, wherein R⁷ and R⁸ are independently selected from the group consisting of C₁-C₁₂-alkyl, C₃-C₁₀-cycloalkyl, aryl, heteroaryl and aralkyl groups, each of which is optionally substituted, or wherein R⁷ and R⁸ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members. When Z is N⁺R⁷R⁸, generally a counter anion A⁻ is present. A⁻ can be selected from the group consisting of Cl⁻, BF₄⁻, PF₆⁻, SbF₆⁻ and AlCl₄⁻, wherein BF₄⁻ and AlCl₄⁻ are preferred, and BF₄⁻ is most preferred. Such compounds are known, for example, from WO2008/022777. R⁷ and R⁸ preferably are selected from the group of C₁ to C₆ alkyl groups, wherein methyl and ethyl are most preferred.

It is preferred that Z is O or N⁺R⁷R⁸, and it is most preferred that Z is O. According to the present invention, in its broadest definition, Y is selected of OR¹³, NR¹⁴R¹⁵ and SR¹⁶, wherein R¹³, R¹⁴, R¹⁵ and R¹⁶ each independently are selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted by one or more substituents of the group S*, or wherein R¹⁴ and R¹⁵ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members. When Y is OR¹³, R¹³ preferably is selected from the group consisting of C₁ to C₄ alkyl groups which is optionally substituted by one or more substituents of the group S*, and more preferably, R¹³ is methyl or ethyl. When Y is SR¹⁶, R¹⁶ preferably is selected from the group consisting of C₁ to C₄ alkyl groups which is optionally substituted by one or more substituents of the group S*, and more preferably, R¹⁶ is methyl or ethyl. When Y is NR¹⁴R¹⁵, which is preferred, R¹⁴ and R¹⁵ preferably are independently selected from the group consisting of C₁ to C₄ alkyl groups which is optionally substituted by one or more substituents of the group S*, or R¹⁴ and R¹⁵ together with the nitrogen atom to which they are bound form an optionally substituted 5- to 10-membered heterocyclic radical which, in addition to the nitrogen atom, wherein a pyrrolidin radical or a piperidine radical is preferred, and which is optionally substituted by one or more substituents of the group S*. In one preferred aspect, Y is NMe₂. In another preferred aspect, Y is a pyrrolidin radical which is attached to the compound of formula (II) though the nitrogen atom.

According to the broadest definition of the present invention, R³ is selected from the group consisting of H, C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted by at least one substituent of the group S* as defined above. In a preferred aspect, R³ is a C₁ to C₄ group, which is optionally substituted by one or more substituent of the group S* as defined above, or an aralkyl group, wherein the aryl group, which is preferably a phenyl group optionally substituted by one or more substituents of the group S*, is attached to the compound of formula (III) or subsequent compounds manufactured from (III) by a C₁-C₈-alkylene chain, which preferably is a —CH₂— or —CH₂—CH₂— chain. Most preferably, R³ is a methyl group.

Generally, R⁵ and R⁶ in (III) and subsequent compounds each independently are selected from the group consisting of H, C₁-C₁₂-alkyl, C₂-C₆ alkenyl, C₃-C₁₀-cycloalkyl, C₂₋₁₂ alkynyl, aryl, heteroaryl or aralkyl group, each of which is optionally substituted by one or more substituents of the group S*, wherein at least one of R⁵ and R⁶ is different from H. Preferably, R⁵ and R⁶ in each independently are selected from the group consisting of C₁ to C₄ alkyl group, H and aryl. In one more preferred aspect, R⁵ is H and R⁶ is phenyl. In another more preferred aspect, R⁵ is methyl and R⁶ is methyl. Each of the foregoing, except H, is optionally substituted by one or more substituents of the group S*.

According to its broadest definition of the present invention, R⁴ in (II), and compounds manufactured from (II) or from which (II) is manufactured, is selected from the group consisting of H, X′, COOR′, OR′, SR′, C(O)NR′₂, wherein the groups R′ are selected independently in C(O)NR′₂ where R′ is selected from the group consisting of hydrogen, C₁-C₁₂-alkyl, CN, C₁-C₁₂-alkyl, C₂-C₆ alkenyl, aryl, cycloalkyl, aralkyl or heteroaryl, each of which is optionally substituted by one or more substituents of the group S* as defined above, and wherein X′ is a halogen atom, wherein X′ is generally selected from the group consisting of F, Cl, Br, and I. Preferably, R⁴ is H or X′. More preferably, R⁴ is selected from the group consisting of H, F and Br, wherein H is most preferred.

The compound of formula (III) can be used in the free hydrazone form, or in the form of a salt, such as a hydrochloride salt. The compound of formula (III) can be, for example, 1-benzylidene-2-methylhydrazine or 1-benzylidene-2-methylhydrazine hydrochloride. As defined above, when (III) is used in its salt form, the amine salt present in the reaction of (II) and (III) is different from (III). The hydrazones of the formula (III) have been described in the literature (Zhurnal Organicheskoi Khimii (1968), 4(6), 986-92.) and can be obtained by reacting commercially available hydrazines with carbonyl compounds.

Typically, the reaction of the compound of formula (II) with the compound of formula (III) is effected in an inert organic solvent. Examples of inert organic solvents are especially aprotic organic solvents such as aromatic hydrocarbons and halohydrocarbons, for example benzene, toluene, xylenes, cumene, chlorobenzene and tert-butylbenzene, cyclic or acyclic ethers such as diethyl ether, diisopropyl ether, tert-butyl methyl ether (MTBE), tert-butyl ethyl ether, tetrahydrofuran (THF) or dioxane, carboxylic acid esters, such as ethyl acetate or isopropyl acetate, nitriles such as acetonitrile and propionitrile, aliphatic halohydrocarbons such as dichloromethane, dichloroethane, trichloromethane and mixtures thereof. It can be advantageous to operate the reaction between the compound of formula (II) and (III) under essentially anhydrous conditions, i.e. the water content in the solution is below 1%, especially below 0.1%, based on the total weight of the solvent.

The compounds of the formula II generally are reacted with the hydrazone of the formula III according to the invention at temperatures in the range from 0 to 180° C., preferably in the range from 10 to 150° C.

Particularly preferred combinations of residues for the reaction of (II) and (III) to obtain (I) are summarized in table 1. The depicted combinations of R¹, R², R³, R¹⁰, R⁴, Y and Z also apply for R⁵ and R⁶=Me.

TABLE 1
Particularly preferred combinations of
residues for the reaction of (II) and (III) to obtain (I)

| | R¹ | R² | R¹⁰ | R³ | R⁴ | R⁵ | R⁶ | Y | Z |
|---|---|---|---|---|---|---|---|---|---|
| C1 | CF₂H | C(O)R¹⁰ | CCl₃ | CH₃ | H | H | phenyl | NR¹⁴R¹⁵, | O |
| | | | | | | | | R¹⁴, | |
| | | | | | | | | R¹⁵ = Me | |
| C2 | CF₂H | C(O)R¹⁰ | CF₃ | CH₃ | H | H | phenyl | NR¹⁴R¹⁵, | O |
| | | | | | | | | R¹⁴, | |
| | | | | | | | | R¹⁵ = Me | |
| C3 | CF₂H | C(O)R¹⁰ | CH₃ | CH₃ | H | H | phenyl | NR¹⁴R¹⁵, | O |
| | | | | | | | | R¹⁴, | |
| | | | | | | | | R¹⁵ = Me | |
| C4 | CF₂H | C(O)R¹⁰ | CCl₃ | CH₃ | H | H | phenyl | NR¹⁴R¹⁵ 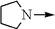 | O |
| C5 | CF₂H | C(O)R¹⁰ | CF₃ | CH₃ | H | H | phenyl | NR¹⁴R¹⁵ 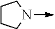 | O |
| C6 | CF₂H | C(O)R¹⁰ | CH₃ | CH₃ | H | H | phenyl | NR¹⁴R¹⁵ 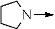 | O |
| C7 | CF₃ | C(O)R¹⁰ | CCl₃ | CH₃ | H | H | phenyl | NR¹⁴R¹⁵, | O |
| | | | | | | | | R¹⁴, | |
| | | | | | | | | R¹⁵ = Me | |
| C8 | CF₃ | C(O)R¹⁰ | CF₃ | CH₃ | H | H | phenyl | NR¹⁴R¹⁵, | O |
| | | | | | | | | R¹⁴, | |
| | | | | | | | | R¹⁵ = Me | |
| C9 | CF₃ | C(O)R¹⁰ | CH₃ | CH₃ | H | H | phenyl | NR¹⁴R¹⁵, | O |
| | | | | | | | | R¹⁴, | |
| | | | | | | | | R¹⁵ = Me | |
| C10 | CF₃ | C(O)R¹⁰ | CCl₃ | CH₃ | H | H | phenyl | NR¹⁴R¹⁵ 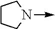 | O |
| C11 | CF₃ | C(O)R¹⁰ | CF₃ | CH₃ | H | H | phenyl | NR¹⁴R¹⁵ 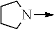 | O |
| C12 | CF₃ | C(O)R¹⁰ | CH₃ | CH₃ | H | H | phenyl | NR¹⁴R¹⁵ 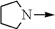 | O |

The invention further concerns a process for the manufacture of a compound of formula (IV), which comprises the process for the manufacture of a compound of formula (I) by reacting a compound of formula (II) and (III), which further comprises the step of contacting the compound of formula (I) with an acid, wherein Z, R¹, R², R³, R⁴, R⁵ and R⁶ in (I) and (IV) are defined as above in general or upstream or downstream compounds.

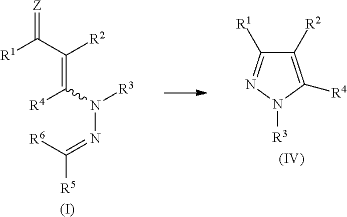

The acid with which the compound of formula (I) is contacted to achieve the formation of the compound of formula (IV) generally is selected from the group consisting of CH₃COOH, H₂SO₄, KHSO₄, NaH₂PO₄, HCl, CF₃SO₃H, CF₃COOH and formic acid. HCl and H₂SO₄ are preferred. Generally, an amount of acid of from 0.01 to 1 equivalents based on the amount of compound of formula (I) is suitable to effect the reaction. It is advantageous that the amount of acid added in the reaction of (I) to cyclize into (IV) is calculated to be sufficient to neutralize any base HNR¹⁴R¹⁵ present from the step of converting the compound of formula (III) into formula (I) in addition to the amount that is intended to effect cyclization of (I) into (IV). Cyclization of (I) generally is carried out at temperatures of from −20° C. to +150° C., preferably at temperatures of from −10° C. to +100° C., particularly preferably at from −10 to 50° C. Often, the reaction is conducted under atmospheric pressure. Suitable solvents for the reaction are, for example, aliphatic, alicyclic or aromatic hydrocarbons, such as, for example, petroleum ether, n-hexane, n-heptane, cyclohexane, methylcyclohexane, benzene, toluene, xylene or decaline, and halogenated hydrocarbons, such as, for example, chlorobenzene, dichlorobenzene, dichloromethane, chloroform, carbon tetrachloride, dichloroethane or trichloroethane, ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole; nitriles, such as acetonitrile, propionitrile, n- or isobutyronitrile or benzonitrile; ketones, such as acetone, methyl ethyl ketone, methyl isobutyl ketone or cyclohexanone; amides, such as N,N-dimethylformamide, N,N-dimethylacetamide, N-methylformanilide, N-methylpyrrolidone or hexamethylphosphoric triamide; sulphoxides, such as dimethyl sulphoxide, or sulphones, such as sulpholane; alcohols, such as methanol, ethanol, n- or isopropanol, butanol; carboxylic acid esters, such as ethyl acetate or isopropyl acetate.

The invention further concerns a process for the manufacture of a compound of formula (V), which comprises the process for the manufacture of (IV) as defined above,

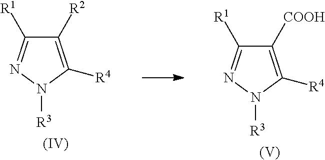

wherein R¹, R², R³ and R⁴ are defined as above in general or upstream compounds (I), (II), (III) or (IV) or subsequent downstream compounds, which comprises at least one of the steps of
a) when R² is C(O)R¹⁰ and R¹⁰ is selected from the group consisting of CX₃, C₂X₅, n-C₃X₇ or iso-C₃X₇, n-, iso- or tert-C₄X₉, wherein in all of the foregoing groups X is the same or different, selected from the group consisting of F, Cl, Br and I, the compound of formula (IV) is contacted with an aqueous base
b) when R² is C(O)R¹⁰ and R¹⁰ is selected from the group consisting of C₁-C₁₂-alkyl, optionally substituted C₃-C₁₀-cycloalkyl, optionally substituted aryl, optionally heteroaryl or optionally substituted aralkyl group, the compound of formula (IV) is contacted with an oxidation agent
c) wherein R² is CN or C(O)OR⁹, the compound of formula (IV) is contacted with an acid or a base.

When the process for the manufacture of formula (V) comprises the step a), R¹⁰ often is selected from the group consisting of CCl₃, C₂Cl₅, n-C₃Cl₇ or iso-C₃Cl₇, n-, iso- or tert-C₄Cl₉, CBr₃, C₂Br₅, n-C₃Br₇ or iso-C₃Br₇, n-, iso- or tert-C₄Br₉. Preferably, R¹⁰ is CCl₃ or CBr₃, and most preferably, R¹⁰ is CCl₃. In step a), (IV) is contacted with a base. The base is an aqueous base. The base is preferably selected from alkali metal or alkaline earth metal bases, such as sodium hydroxide, potassium hydroxide or calcium hydroxide, alkali metal and alkaline earth metal oxides, such as lithium oxide, sodium oxide, calcium oxide or magnesium oxide, alkali metal and alkaline earth metal carbonates, such as lithium carbonate or calcium carbonate, alkali metal bicarbonates, such as sodium bicarbonate, alkali metal and alkaline earth metal hydrides, such as lithium hydride, sodium hydride, potassium hydride or calcium hydride, or alkali metal amides, such as lithium amide, sodium amide or potassium amide. Hydroxides of alkali metal or alkaline earth metals are preferred. Step a) generally is assumed to proceed via an intermediate of formula (Vi)

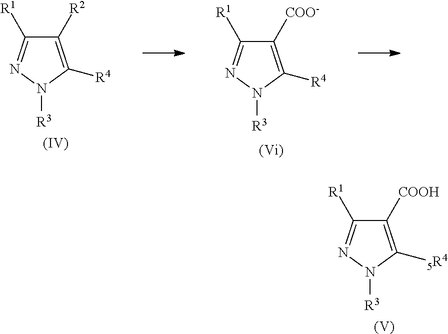

In order to obtain the compound of formula (V) from the compound of formula (IV), after the compound of formula (IV) is contacted with an aqueous base, often the intermediate (Vi) is contacted with at least one acid in order to obtain the compound of formula (V). The at least one acid can be selected, for example, inorganic acids such as hydrochloric acid, sulphuric acid, nitric acid, phosphoric acid and organic acids such as trifluoroacetic acid, methane sulphonic acid and para-toluene sulphonic acid.

When R¹⁰ is selected from the group consisting of CH₍₃₋ₘ₎Xₘ wherein m=0-2, C₂H₍₅₋ₘ₎Xₘ wherein m=0-4, n- or iso-C₃H₍₇₋ₘ₎Xₘ wherein m=0-6, n-, iso- or tert-C₄H₍₉₋ₘ₎Xₘ wherein m=0-8, the process for the manufacture of (V) can compose a step wherein (IV) is contacted with a halogenating agent. The halogenating agent often is selected from the group consisting of a hypohalite, a base B and a halide, a halide, such as F₂, Cl₂, Br₂ and I, mixed (interhalogen) halides, such as BrCl, ClF, ClF, ICl, N-halosuccinimide, such as N-fluorosuccinimide, N-bromoosuccinimide, N-chlorosuccinimide and N-iodosuccinimide, thionyl halide, such as thionyl fluoride, thionyl bromide, thionyl chloride and thionyl iodide, phosphorous trihalide, such as PCl₃, PBr₃, PI₃, phosphorous pentahalide, such as PCl₅, PBr₅, Et₃N.3HF (TREAT-HF), (HF)ₓ.Pyr (Olahs reagent), Et₂NSF₃ (DAST), (Me₂N)₃S(Me)₃SiF₂ (TASF), PhIF₂, BF₃, XeF₂, CH₃COOF, CF₃COOF, CF₃OF, FOClO₃, N-Fluorobenzene-sulfonimide chloride and sulfuryl chloride. The term “hypohalite” intends to denotes a hypohalous acid HOX or salts thereof, wherein the anion is selected from BrO⁻, FO⁻, IO⁻ and ClO⁻, and the cation is an alkali or earth alkali cation. Often, the hypohalite is selected from NaOCl, Ca(OBr)₂, NaOBr and Ca(ClO)₂. The combination “a base B and a halide” intends to denote a combination of F₂, Cl₂, Br₂ and I₂ with an aqueous inorganic base B, such as alkali hydroxide or earth alkali hydroxide, or an organic base B, such as NEt₃. By this reaction, the number of halogen atoms X on the group R¹⁰ can be increased from its initial number, for example from CH₃ to CHX₂, from CH₃ to CH₂X, from CH₃ to CX₃, from CHX₂ to CX₃, from C₂H₅ to partially or fully halogenated ethyl, from iso-propyl to partially or fully halogenated n-propyl, from iso-propyl to partially or fully halogenated n-propyl, and from n-, iso- or n-butyl to partially or fully halogenated n-, iso- or n-butyl. When full halogenation is achieved, step a) can be applied.

When the process for the manufacture of formula (V) comprises the step b), R² is C(O)R¹⁰ and R¹⁰ is selected from the group consisting of C₁-C₁₂-alkyl, optionally substituted C₃-C₁₀-cycloalkyl, optionally substituted aryl, optionally heteroaryl or optionally substituted aralkyl group, and the compound of formula (IV) is contacted with an oxidation agent. R¹⁰ preferably is selected from the group of C₁ to C₄ alkyl groups, and is most preferably methyl. Oxidation agents are not particularly limited and include, for example, halogens, such as chlorine, bromine, iodine; oxo acids of halogens and salts thereof, such as hypochlorous acid and salts thereof, hypobromous acid and salts thereof, chlorous acid and salts thereof, iodic acid and salts thereof, periodic acid and salts; peroxides such as hydrogen peroxide; and molecular oxygen. Examples of oxo acid salt counter cations include Na⁺, K⁺, ½Ca²⁺, NH⁺ and the like. When the oxidation agent is oxygen, preferably a catalyst is present, such as a metal containing catalyst, for example oxides, nitrates, acetates, halides, or hydrates of Ti, Zr, V, Nb, Ta, Cr, Mo, W, Mn, Tc, Re, Fe, Ru, Ir, Ni, Pd, Pt, Cu, Ag, Au, Zn, Cd and Hg. Preferred metal catalysts include Fe(NO₃)₃ or its hydrate, Co(NO₃)₃ or its hydrate, Ni(NO₃)₃ or its hydrate, Co(NO₃)₃ or its hydrate, Mn(NO₃)₃ or its hydrate, Zn(NO₃)₃ or its hydrate, Mn(OAc₂), Co(OAc₂), Cu(OAc₂), CuCl₂ or its hydrate, CuO, CuBr₂, CuCl, CuBr, CuI and Re₂O₇.

As oxidants, an oxidant containing chlorine is preferred, hypochlorous acid or salts thereof are more preferred and hypochlorous acid is particularly preferred.

The oxidation reaction of step b) can be carried out under acidic, neutral and basic conditions. Under basic conditions, for example, if a basic oxidant is used as an oxidant or if an oxidation reaction is carried out under basic conditions, a carboxylate of compound (V), which is the compound of formula (Vi) as described above, is formed, which can be transformed into (V) by contact with an acid. The at least one acid can be selected, for example, from inorganic acids such as hydrochloric acid, sulphuric acid, nitric acid, phosphoric acid and organic acids such as trifluoroacetic acid, methane sulphonic acid and para-toluene sulphonic acid.

Examples of bases which can be present in step b) in the oxidation of compound (IV) into (V) include inorganic bases and organic bases, in particular when a halogen is the oxidation agent. Examples of inorganic bases include alkali metal and alkaline earth metal hydroxides such as sodium hydroxide, potassium hydroxide, calcium hydroxide and the like; alkali metal and alkaline earth metal oxides such as lithium oxide, sodium oxide, calcium oxide, magnesium oxide and the like; alkali and alkaline earth metal carbonates such as lithium carbonate, calcium carbonate and the like; alkali and alkaline earth metal bicarbonates such as sodium bicarbonate, potassium bicarbonate and the like; alkali and alkaline earth metal hydrides such as lithium hydride, sodium hydride, potassium hydride, calcium hydride; and alkali metal amides such as lithium amide, sodium amide, potassium amide and the like. Examples of organic bases include amines such as triethylamine, dimethylamine and the like and ammonia. As bases, alkali or alkaline earth metal hydroxides, alkali or alkaline earth metal oxides, alkali or alkaline earth metal carbonates, and alkali metal bicarbonates are preferred, alkali or alkaline earth metal hydroxides are further preferred and sodium hydroxide, potassium hydroxide and calcium hydroxide are most preferred.

When the process for the manufacture of a compound of formula (V) comprises the step c), R² is CN or C(O)OR⁹, and the compound of formula (IV) is contacted with an acid or a base. When R² is C(O)OR⁹, R⁹ preferably is selected from the group of C₁ to C₄ alkyl groups, wherein methyl and ethyl are most preferred. The conversion of the group CN to carboxylic acids with acids or bases is known to the person skilled in the art, for example from Houben-Weyl, Methods of Organic Chemistry, Vol. II, 4^{th} Edition, 1953, p. 533-535. The conversion of the group C(O)OR⁹ to carboxylic acids with acids or bases is known to the person skilled in the art.

The compounds of formula (V) are important intermediates for the manufacture of pharmaceutically or agrochemically active compounds, in particular carboxamides of the class of SDHI fungicides, notably Sedaxane, Fluopyram, Benzovindiflupyr, Bixafen, Fluxapyroxad, Isopyrazam, Penflufen and Penthiopyrad.

Another object of the present invention is a process for the manufacture of a compound of formula (VI), which comprises the process for the manufacture of a compound of formula (V), and which further comprises a first step, wherein the compound of formula (V) is reacted with a halogenating agent, an acylating agent or CDI (carbonyldiimidazole), and a second step, wherein the product from the first step is contacted with a compound of formula (VII) NHR¹⁷Q, wherein R¹⁷ is selected from the group consisting of H, C₁-C₁₂-alkyl, C₂-C₆ alkenyl or C₃-C₈-cycloalkyl group, wherein H and C₁-C₄-alkyl are preferred, and wherein Q is an optionally substituted aryl or heteroaryl group.

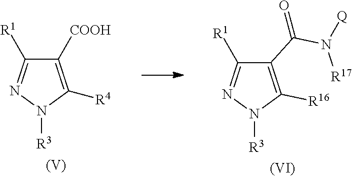

When the compound of formula (V) is reacted with a halogenating agent, the halogenating agent often is selected from the group consisting of oxalyl chloride, thionyl chloride, phosphorous trichloride, PCl₅, POCl₃ and COCl₂ and phosphorous pentachloride, Ph₃P and CCl₄ and cyanuric chloride. The compound of formula (V) is transformed in this first step into a carboxylic acid halide, which is then reacted in a second step with the compound of formula (VII).

When the compound of formula (V) is reacted with a acylating agent, suitable acylating agents generally include carboxylic acid anhydrides, such as acetic acid anhydride and trifluoroacetic acid anhydride, and carboxylic acid halides, such as trifluoroacetyl chloride. In the reaction of the compound of formula (V) with an acylating agent, presence of a base, such as, for example, triethylamine, can be advantageous.

The activation of carboxylic acids with CDI, as can be applied to the compound of formula (V) in a first step prior to the reaction with the compound of formula (VII) is described, for example, in E. K. Woodman et al, Org. Process Res. Dev., 2009, 13 (1), pp 106-113.

In the compound of formula (VII) NHR¹⁷Q, R¹⁷ preferably is methyl, ethyl, cyclopropyl or H, wherein H is most preferred.

Generally, Q is an aryl or heteroaryl group, which can be optionally substituted by one or more substituents of the group S* as defined before. More specifically, Q can be an optionally substituted aromatic carbocycle, non-aromatic or aromatic heterocyclic group, all of which can also be bi- or tricyclic, wherein one or more rings which are bound to the aromatic carbocycle or heterocyclic group can be non-aromatic. Often, Q is selected from the group consisting of phenyl, naphtalene, 1,2,3,4-tetrahydronaphthalene, 2,3-dihydro-1H-indene, 1,3-dihydroisobenzofuran, 1,3-dihydrobenzo[c]thiophene, 6,7,8,9-tetrahydro-5H-benzo[7]annulene, thiophene, furan, thiazole, thiadiazole, oxazole, oxadiazole, pyridine, pyrimidine, triazine, tetrazine, thiazine, azepine and diazepine, each of which is optionally substituted by one or more substituents of the group S* as defined before. In one aspect, Q is a group of formula Q1

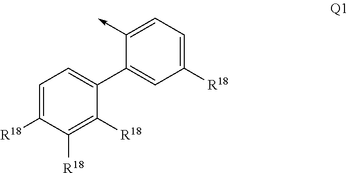

wherein each R¹⁸ is independently selected from the group consisting of hydrogen or halogen, said halogen is especially chlorine or fluorine. In particular, Q1 is the residue 3′,4′-dichloro-5-fluorobiphenyl-2-yl or the residue 3′,4′,5′-trifluorophenyl)phenyl.

In another aspect, Q is a group of formula Q2

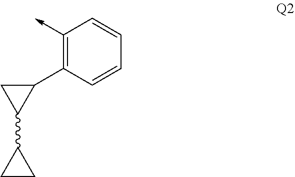

In another aspect, Q is a group of formula Q3, including all its stereoisomers:

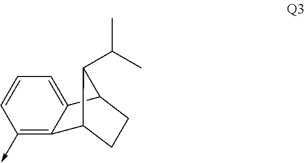

In yet another aspect, Q is a group of formula Q4

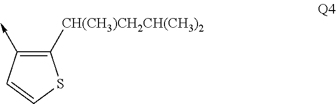

In a further aspect, Q is a group of formula Q5, including all of its stereoisomers, wherein R¹⁹ is H or halogen, in particular R¹⁹ is Cl.

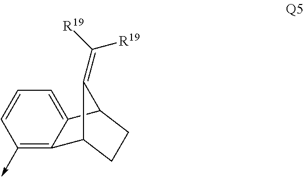

In the reaction of the product of the reaction of the compound of formula (V) with a halogenating agent, an acylating agent or CDI, it can be advantageous when a base such as triethylamine is present.

The invention further concerns a process for the manufacture of a compound of formula (VI), which comprises the process for the manufacture of the compound of formula (IV) as described above, and which further comprises one of the steps d) and e), wherein in
d) when R² in the compound of formula (IV) is C(O)R¹⁰, R¹⁰ is selected in the compound of formula (IV) is selected from the group consisting of CX₃, C₂X₅, n-C₃X₇ or iso-C₃X₇, n-, iso- or tert-C₄X₉, wherein in all of the foregoing groups X is the same or different, selected from the group consisting of F, Cl, Br and I, the compound of formula (IV) is contacted with a compound of formula NHR¹⁷Q, wherein R¹⁷ and Q are defined as above or
e) when R² in the compound of formula (IV) is C(O)OR⁹, wherein R⁹ is defined above, the compound of formula (IV) is contacted with a compound of formula NHR¹⁷Q, wherein R¹⁷ and Q are defined as above, and at least one compound selected of the group consisting of a Lewis acid or a base.

When the process for the manufacture of a compound of formula (VI) comprises the step d), R¹⁰ in the compound of formula (IV) often is selected from the group consisting of CCl₃, C₂Cl₅, n-C₃Cl₇ or iso-C₃Cl₇, n-, iso- or tert-C₄Cl₉, CBr₃, C₂Br₅, n-C₃Br₇ or iso-C₃Br₇, n-, iso- or tert-C₄Br₉. Preferably, R¹⁰ is CF₃, CCl₃ or CBr₃, and most preferably, R¹⁰ is CCl₃. The principles of the reaction of such a compound (IV) with the compound of formula (VII) NHR¹⁷Q are described in WO2017/129759, which is hereby incorporated by reference for all purposes.

When the process for the manufacture of a compound of formula (VI) comprises the step e), when R² in the compound of formula (IV) is C(O)OR⁹, wherein R⁹ is defined as above and is preferably methyl or ethyl, the compound of formula (IV) is contacted with a compound of formula NHR¹⁷Q, wherein R¹⁷ and Q are defined as above, and at least one compound selected of the group consisting of a Lewis acid or a base. The principles of the reaction of such a compound (IV) with the compound of formula (VII) NHR¹⁷Q in the presence of a base or a Lewis Acid are described in WO2012055864 and WO2016/016298, which are hereby incorporated by reference for all purposes.

The compounds of formula (II) are well established. For example, the compounds of formula (II), wherein R² is the group C(O)R¹⁰, can be obtained by reaction of a compound of formula (VIII) with a compound of formula (IX) or (X), wherein X″ in the compound of formula (IX) is selected from F, Cl and Br, an preferably is F or Cl. Z, R¹, R⁴, R¹⁰ and Y are the same as defined before for any of compounds (I) to (VI)

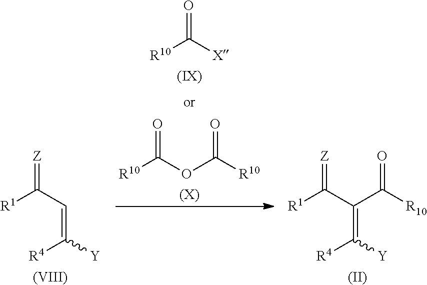

Compounds of formula (II) wherein Y is the group NR¹⁴R¹⁵ can be obtained from compound of formula (II) wherein OR¹³ by reacting the compound of formula (II) wherein OR¹³ with the respective amine HNR¹⁴R¹⁵.

Compounds of formula (VIII) are also well known in the prior art, for example 4-ethoxy-1,1,1-trifluoro-3-buten-2-one (ETFBO), which can be obtained through methods described for example in WO2010000871, which is hereby incorporated by reference for all purposes, or (4-ethoxy-1,1,1-trichloro-3-buten-2-one ETCBO), which can be obtained through methods described for example in Tietze, L. F. et al, Organic Syntheses, 69, 238-244; 1990. Compounds of formula (II) wherein Z is N′R⁷R⁸ can be obtained from the addition of reagents obtained from, for example, the reaction of 1,1,2,2-tetrafluoro-N,N-dimethylethanolamine and a Lewis Acid such as BF₃, as described in WO2016152886.

The compounds of formula (IX) are carboxylic acid halides. Many compounds falling under the formula (IX) are well established and commercially available. The manufacture of difluoroacetyl fluoride is, for example, disclosed in EP694523 and U.S. Pat. No. 5,905,169 which are hereby incorporated by reference for all purposes. The manufacture of difluorochloroacetyl chloride is, for example, disclosed in U.S. Pat. No. 5,545,298 or 5,569,782, which are hereby incorporated by reference for all purposes, as well as the manufacture of trifluoroacetylchloride. The manufacture of carboxylic acid anhydrides such as (X) is known, for example, from WO2014195929, which is hereby incorporated by reference for all purposes. The step to manufacture compound (II) from compound (VIII) generally is performed in the presence of a suitable solvent or a mixture of suitable solvents. Suitable solvents are, for example, nonpolar aprotic solvents, for example aromatic hydrocarbons, such as benzene, toluene, xylenes, or (cyclo)aliphatic hydrocarbons, such as hexane, cyclohexane and the like, and also mixtures of the solvents mentioned above. Examples of suitable organic solvents are likewise aprotic polar solvents, for example cyclic and acyclic ethers, such as diethyl ether, tert-butyl methyl ether (MTBE), diisopropyl ether, cyclopentyl methyl ether, tetrahydrofuran (THF) or dioxane, cyclic or acyclic amides, such as dimethyl formamide, dimethyl acetamide, N-methylpyrrolidone, ureas, such as N,N′-dimethyl-N,N′-ethyleneurea (DMEU), N,N′-dimethyl-N,N′-propyleneurea (DMPU) or tetramethylurea, or aliphatic nitriles, such as acetonitrile or propionitrile. Halogenated hydrocarbon solvents, such as chloroform or dichloromethane, can also be suitable solvents. Ethylacetate, toluene, dichloromethane and chloroform are preferred solvents.

In another aspect, the step to manufacture compound (II) from compound (VIII) can be performed in the absence of a solvent.

In one aspect, the step to manufacture compound (II) wherein R² is C(O)R¹⁰ from compound (VIII) is performed in the presence of at least one base, which is preferred. This base is different from the base of formula (VIII) when Y═NR¹⁴R¹⁵. Particularly suitable are organic cyclic and acyclic aromatic or non-aromatic bases, such as triethylamine, diisopropylamine, pyridine, pyrimidine, trimethylamine, tributylamine, diisopropylethylamine, tert-butyldimethylamine, N-methylpyrrolidine, N-methylpiperidine, N-methylmorpho line, N,N′-dimethylpiperazine, collidine, lutidine or 4-dimethylaminopyridine, and bicyclic amines, such as diazabicycloundecene (DBU) or diazabicyclononene (DBN). Inorganic bases are also suitable as bases to be present in the step to manufacture compound (II) from compound (VIII), for example alkali metal and alkaline earth metal carbonates, such as lithium carbonate or calcium carbonate, alkali metal bicarbonates, such as sodium bicarbonate, alkali metal and alkaline earth metal oxides, such as lithium oxide, sodium oxide, calcium oxide or magnesium oxide, alkali metal and alkaline earth metal hydrides, such as lithium hydride, sodium hydride, potassium hydride or calcium hydride, or alkali metal amides, such as lithium amide, sodium amide or potassium amide. Neutral organic bases, such as DMF or acetamides are particularly suitable as base. The presence of a base in particularly advantageous when Y═NR¹⁴R¹⁵ in (VIII).

In a one aspect of the process according to the invention for preparing compounds of the formula (II) wherein R² is C(O)R¹⁰ from a compound of formula (VIII) is carried out essentially anhydrously.

For the present invention, the term «essentially anhydrous» in intended to denote that a solvent, reagent, reaction mixture and/or additive has a water content of less than 500 ppm and in particular of less than 100 ppm. The water released during the reaction is not taken into account in the stated water content. The step to manufacture compound (II) from the compound of formula (VIII) is often performed at a temperature which generally is equal to or greater than −80° C., preferably equal to or greater than −70° C. and more preferably equal to or greater than −60° C. Often, the temperature is equal to or less than 80° C., preferably equal to or less than 60° C. and more preferably equal to or less than 40° C.

In one preferred aspect of the present invention, the compound of formula (II) wherein R² is C(O)R¹⁰ is manufactured from the compound of formula (VIII) with a compound of formula (IX) in the presence of a base. The base is the amine from which the amine salt according to the present invention, which is present in the manufacture of the compound of formula (I), derives. Suitable amines are described in the section defining suitable amines from which the amine salt derives; particularly suitable amines are pyridine and triethylamine. The amine which is present as a base in the reaction of (VIII) with (IX) forms an amine salt with the X″ of the compound of formula (IX), which is preferably Cl or F. For example, pyridine hydrochloride, pyridine hydrofluoride, triethylamine hydrochloride or triethylamine hydrofluoride is formed. The amine salt formed in the reaction of the compound of formula (VIII) with the compound of formula (XI) advantageously, in a preferred aspect of the invention, is not removed from the mixture comprising the compound of formula (II), but remains in the mixture comprising (II), thus being transferred as amine salt to the reaction of the compound of formula (II) with the compound of formula (III) to obtain the compound of formula (I). Thus, the invention concerns a process for the manufacture of a compound of formula (I), which comprises the step of reaction of a compound of formula (II) with a compound of formula (III) in the presence of an amine salt, and which further comprises a step wherein a compound of formula (VIII) is reacted with a compound of formula (IX) in the presence of a base which is the amine from which the amine salt present in the step of manufacturing the compound of formula (I) derives so that the compound of formula (II) is obtained with a co-product which is the amine salt.

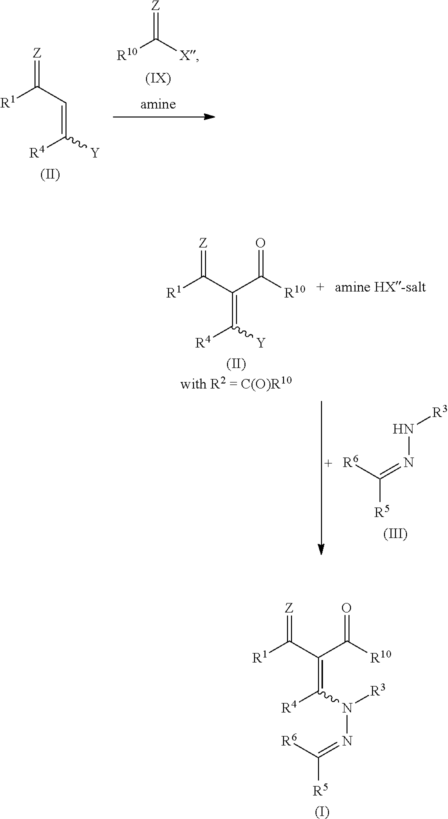

The process for the manufacture of the compound (I) and optionally upstream and downstream steps according to the present invention allow for efficient manufacture of the compound of formula (I) and its downstream products (IV), (V) and (VI) which are intermediates for active ingredients in the agrochemical or pharmaceutical field or are themselves active ingredients in the agrochemical or pharmaceutical field. The process not only proceeds with high yields, thus making possible the efficient manufacture of the products, but also avoids waste, or even makes use of waste in the form of co-product amine salt from an upstream step. It is possible to conduct the process for the manufacture of compound (I) under relatively mild conditions, avoiding e.g. acidic catalysts or auxiliaries known from the prior art which might lead to decomposition of the desired product.

Should the disclosure of any patents, patent applications, and publications which are incorporated herein by reference conflict with the description of the present application to the extent that it may render a term unclear, the present description shall take precedence.

The following examples are intended to further explain the invention without limiting it.

### EXAMPLE 1 1,1,1-TRICHLORO-4-ETHOXYBUT-3-EN-2-ONE (ATCBO)

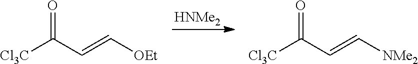

1,1,1-trichloro-4-ethoxybut-3-en-2-one (ETCBO, 0.46 mol) is dissolved in in 150 mL toluene. To this mixture, 21.7 g (0.48 mol) of dimethylamine gas are added. The mixture is stirred for 3 hours at room temperature. Full conversion into 1,1,1-trichloro-4-(dimethylamino)-but-3-en-2-one (ATCBO) is monitored by GC. The mixture is transferred into a 1 liter flask, and the volatiles are partially removed. The remaining liquid contains toluene, EtOH and ATCBO. The mixture is used without further purification in the next step.

### EXAMPLE 2 (1,1,1-TRICHLORO-3-((DIMETHYLAMINO)-METHYLENE)-5,5-DIFLUOROPENTANE-2,4-DIONE

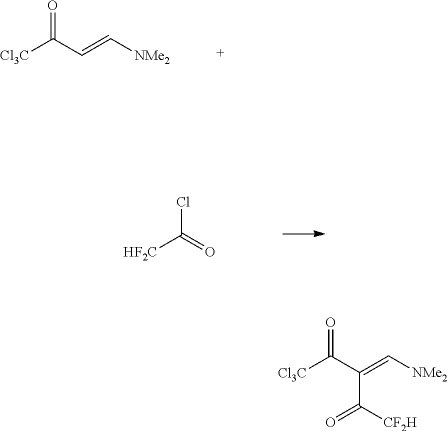

The mixture obtained in example 1, containing 0.46 mol ATCBO, is diluted with 200 mL toluene. 44.3 mL pyridine are added (0.55 mol, 1.2 eq), and 65.19 g (0.55 mol) 2,2-difluoroacetyl chloride are added via syringe under the solvent level. The mixture is stirred at 60° C. for 4 hours until ¹H-NMR shows full conversion.

### EXAMPLE 3 3-((2-(BENZYLIDENE)-1-METHYLHYDRAZINYL)METHYLENE)-1,1,1-TRICHLORO-5,5-DIFLUOROPENTANE-2,4-DIONE

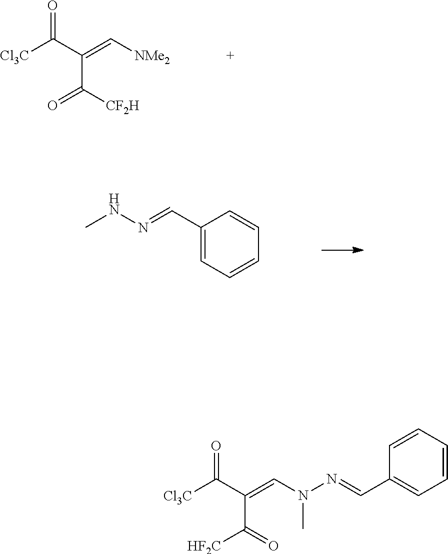

70 g of 1-benzylidene-2-methylhydrazine, obtained by reaction of benzaldehyde and methylhydrazine, are added to the mixture obtained in example 2, and the mixture is stirred for 3 hours at 60° C.

### EXAMPLE 4 2,2,2-TRICHLORO-1-(3-(DIFLUOROMETHYL)-1-METHYL-1H-PYRAZOL-4-YL)ETHAN-1-ONE

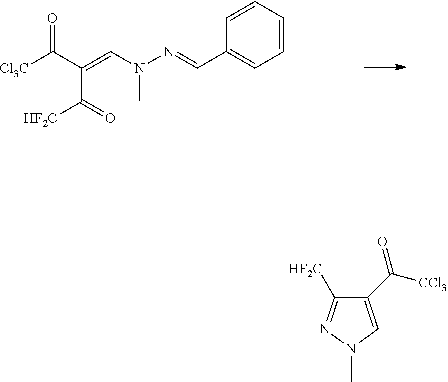

To the mixture of example 3, 32 mL of conc. H₂SO₄ are added dropwise at 60° C. After completed addition, the mixture is stirred for another 2 hours at 60° C. until ¹H-NMR monitoring shows full conversion. The mixture is cooled to room temperature. 25 mL of water are added. The aqueous phase is separated, extracted twice with toluene, and the organic phases are combined. The combined organic phases are used in the next step.

### EXAMPLE 5 3-(DIFLUOROMETHYL)-1-METHYL-1H-PYRAZOLE-4-CARBOXYLIC ACID

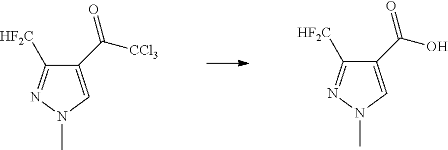

To the mixture from example 4, 26.2 g of NaOH in 80 mL water are added. The mixture is heated to 60° C. for 2 hours. Full conversion is monitored by ¹H-NMR. The phases are separated and the aqueous phase extracted with 40 mL toluene. The aqueous phase is acidified with 32% aq HCl (66 mL) under vigorous stirring. The suspension which forms is cooled under stirring to 0° C., filtered and washed with cold water (3 times 60 mL water). The wet cake is dried under air stream for several hours at room temperature to yield the product.

### EXAMPLE 6 3-(DIFLUOROMETHYL)-1-METHYL-1H-PYRAZOLE-4-CARBOXYLIC ACID CHLORIDE

3-(difluoromethyl)-1-methyl-1H-pyrazol-4-carboxylic acid obtained by example 5 is treated with oxalyl chloride (1.25 eq) in toluene, and a few drops of dimethylformamide are added. The mixture is concentrated under reduced pressure to yield the carboxyl chloride.

### EXAMPLE 7 BIXAFEN

(1.3 mmol) 3′,4′-dichloro-5-fluoro-1,1′-biphenyl-2-amine and (1.56 mmol) 3-(difluorochloromethyl)-1-methyl-1H-pyrazol-4-carboxylic acid chloride obtained by Example 6 are solved in 6 ml tetrahydrofuran and mixed with 2.6 mmol triethylamin. The mixture is stirred for 16 h at 60° C. The mixture is concentrated and chromatographed on silica using cyclohexane/acetic acid ethyl ester to yield bixafen.

### EXAMPLE 8: FLUXAPYROXAD (3-(DIFLUOROMETHYL)-1-METHYL-N-(3′,4′,5′-TRI-FLUOROBIPHENYL-2-YL)-1H-PYRAZOLE-4-CARBOXAMIDE)

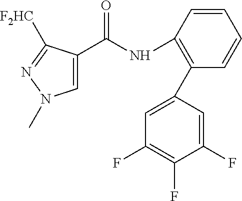 [SMILES: Cn1cc(C(=O)Nc2ccccc2-c2cc(F)c(F)c(F)c2)c(C(F)F)n1]

Fluxapyroxad is obtained using the procedure of example 7, wherein 3′,4′,5′-trifluorobiphenyl-2-amine is used instead of 3′,4′-dichloro-5-fluorobiphenyl-2-amine.

### EXAMPLE 9: SEDAXANE (N-(2-(BI(CYCLOPROPAN)-2-YL)PHENYL)-3-(DIFLUOROME-THYL)-1-METHYL-1H-PYRAZOLE-4-CARBOXAMIDE)

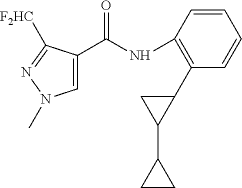 [SMILES: Cn1cc(C(=O)Nc2ccccc2C2CC2C2CC2)c(C(F)F)n1]

Sedaxane is obtained using the procedure of example 7, wherein 2-(bi(cyclopropan)-2-yl)aniline is used instead of 3′,4′-dichloro-5-fluorobiphenyl-2-amine.

Additional experiments can be carried out under similar conditions with respectively hexanes, tetrahydrofuran and ethyl acetate as solvent. Additional experiments can be carried out under similar conditions with respectively toluene, hexanes, ethyl acetate and isopropyl acetate as solvent and R′═CF₃.

### EXAMPLE 10: 1,1,1-TRICHLORO-4-ETHOXYBUT-3-EN-2-ONE (ATCBO)

1,1,1-trichloro-4-ethoxybut-3-en-2-one (ETCBO, 0.46 mol) was dissolved in in 200 mL i-propyl-acetate. To this mixture, 21.7 g (0.48 mol) of dimethylamine gas were added at room temperature. A mild exothermicity was observed. The mixture was stirred for 3 hours at room temperature. Full conversion into 1,1,1-trichloro-4-(dimethylamino)-but-3-en-2-one (ATCBO) was monitored by ¹H-NMR. The mixture was transferred into a 1 liter flask, and the volatiles were partially removed at 500 mbar/80° C. The remaining liquid contained toluene, EtOH and ATCBO. The mixture was used without further purification in the next step.

### EXAMPLE 11 (1,1,1-TRICHLORO-3-((DIMETHYLAMINO)-METHYLENE)-5,5-DIFLUOROPENTANE-2,4-DIONE

The mixture obtained in example 10, containing 0.46 mol ATCBO, was diluted with 300 mL i-propyl-acetate. 44.3 mL pyridine were added (0.55 mol, 1.2 eq), and 65.19 g (0.55 mol) 2,2-difluoroacetyl chloride were added via syringe under the solvent level. The mixture was stirred at 50° C. for 4 hours until ¹H-NMR showed full conversion.

### EXAMPLE 12 3-((2-(BENZYLIDENE)-1-METHYLHYDRAZINYL)METHYLENE)-1,1,1-TRICHLORO-5,5-DIFLUOROPENTANE-2,4-DIONE

70 g of 1-benzylidene-2-methylhydrazine, obtained by reaction of benzaldehyde and methylhydrazine, were added to the mixture obtained in example 11, and the mixture was stirred for 3 hours at 50° C.

### EXAMPLE 13 2,2,2-TRICHLORO-1-(3-(DIFLUOROMETHYL)-1-METHYL-1H-PYRAZOL-4-YL)ETHAN-1-ONE

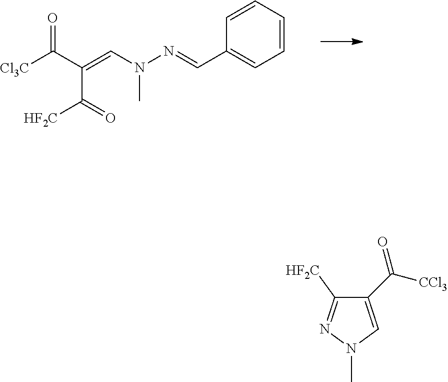

To the mixture of example 12, 32 mL of conc. H₂SO₄ were added dropwise at 50° C. After completed addition, the mixture was stirred for another 2 hours at 50° C. until ¹H-NMR monitoring showed full conversion. The mixture was cooled to room temperature. 25 mL of water were added. The aqueous phase was separated, extracted twice with i-propyl-acetate, and the organic phases were combined. The combined organic phases were used in the next step.

### EXAMPLE 14 3-(DIFLUOROMETHYL)-1-METHYL-1H-PYRAZOLE-4-CARBOXYLIC ACID

To the mixture from example 13, 26.2 g of NaOH in 80 mL water were added. The mixture was heated to 50° C. for 2 hours. Some crystallization occurred, whereupon 20 mL of water were added. An additional 6 g of NaOH in 15 mL water were added to drive full conversion. Full conversion was monitored by ¹H-NMR. The phases were separated and the aqueous phase extracted with 40 mL i-propyl-acetate. The aqueous phase was acidified with 32% aq HCl (66 mL) under vigorous stirring. The yellow thick suspension which formed was cooled under stirring to 10° C., filtered and washed with cold water (3 times 60 ml, water). The wet cake was dried under air stream for several hours at room temperature to yield 66.56 g (0.378 mol) of a beige, solid product. The yield 82.12% is the yield calculated on basis of the initial amount ETCBO in example 9 (yield over 5 steps). ¹H-NMR purity was +99%.

1. A process for manufacturing a compound according to formula (I), the process comprising reacting a compound of formula (II) and a compound of formula (III) 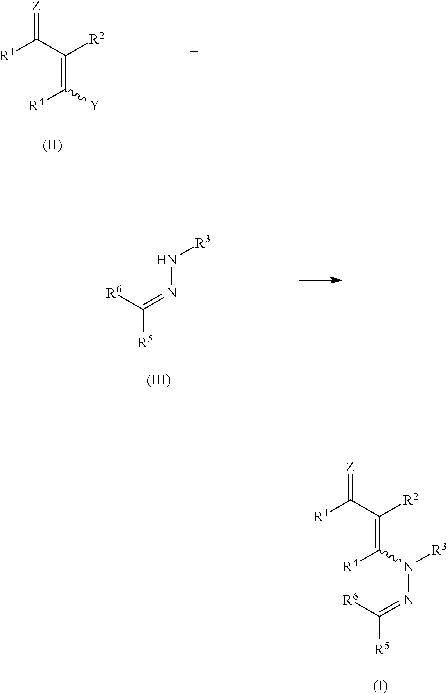
wherein Z is selected from O, S and N⁺R⁷R⁸, wherein R⁷ and R⁸ are independently selected from the group consisting of C₁-C₁₂-alkyl, C₃-C₁₀-cycloalkyl, aryl, heteroaryl and aralkyl groups, each of which is optionally substituted, or wherein R⁷ and R⁸ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members,
wherein R¹ is selected from the group consisting of optionally substituted C₁ to C₄ alkyl groups,
wherein R² is selected from the group consisting of C(O)OR⁹, CN, C(O)R¹⁰ and C(O)NR¹¹R¹², wherein R⁹, R¹⁰, R¹¹ and R¹² each independently are selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted, or wherein R¹¹ and R¹² together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members,
wherein Y is selected of OR¹³, NR¹⁴R¹⁵ and SR¹⁶, wherein R¹³, R¹⁴, R¹⁵ and R¹⁶ each independently are selected from the group consisting of C₁-C₁₂-alkyl, C₂-C₆ alkenyl, C₃-C₁₀-cycloalkyl, C₂-12 alkynyl, aryl, heteroaryl and aralkyl groups, each of which is optionally substituted, or wherein R¹⁴ and R¹⁵ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members,
wherein R³ is selected from the group consisting of H, C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted,
wherein R⁵ and R⁶ each independently are selected from the group consisting of H, C₁-C₁₂-alkyl, C₂-C₆ alkenyl, C₃-C₁₀-cycloalkyl, C₂₋₁₂ alkynyl, aryl, heteroaryl or aralkyl group, each of which is optionally substituted, wherein at least one of R⁵ and R⁶ is different from H,
wherein R⁴ is selected from the group consisting of H, X′, COOR′, OR′, SR′, C(O)NR′₂, wherein the groups R′ are selected independently in C(O)NR′₂ where R′ selected from the group consisting of hydrogen, C₁-C₁₂-alkyl, CN, C₁-C₁₂-alkyl, C₂-C₆ alkenyl, aryl, cycloalkyl, aralkyl and heteroaryl, each of which is optionally substituted, and wherein X′ is a halogen atom,
wherein in the reaction of the compound of formula (I) and (II), an amine salt is present.

2. The process according to claim 1, wherein the anion of the amine salt is a halide anion.

3. The process according to claim 1, wherein the amine salt is derived from a secondary or tertiary amine.

4. The process according to claim 3, wherein the amine salt is a tertiary aromatic amine salt selected from the group consisting of halide salts of 2,6-lutidine, 2-methylpyridine, 3-methylpyridine, 4-methylpyridine or pyridine.

5. The process according to claim 1, wherein R¹ is selected from the group of C₁ to C₄ alkyl groups, wherein the alkyl group is substituted by at least one halogen atom.

6. The process according to claim 5, wherein R¹ is selected from the group consisting of CF₃, CHF₂, CH₂F, CCl₃, CHCl₂, CH₂Cl, CBr₃, CBr₂H, CBrH₂, CI₃, CI₂H, CBr₂Cl, CCl₂Br, C₂F₅, C₂Br₅ and C₂Cl₅.

7. The process according to claim 1, wherein R² is C(O)R¹⁰ and R¹⁰ is selected from the group consisting of C₁-C₁₂-alkyl, C₃-C₁₀-cycloalkyl and aryl groups, each of which is optionally substituted.

8. The process according to claim 1, wherein Z is O or N⁺R⁷R⁸.

9. The process according to claim 1, wherein Y is NR¹⁴R¹⁵ wherein R¹⁴ and R¹⁵ each independently are selected from the group consisting of C₁-C₁₂-alkyl, C₃-C₁₀-cycloalkyl, aryl and aralkyl groups, each of which is optionally substituted, or wherein R¹⁴ and R¹⁵ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members.

10. The process according to claim 1, wherein R³ is selected from the group consisting of H and C₁-C₁₂-alkyl each of which is optionally substituted.

11. The process according to claim 1, wherein R⁴ is selected from the group consisting of H and X′.

12. A process for the manufacture of a compound of formula (IV), which comprises the process according to claim 1, and which further comprises the step of contacting the compound of formula (I) with an acid, wherein Z, R¹, R², R³, R⁴, R⁵ and R⁶ are defined as in claim 1.

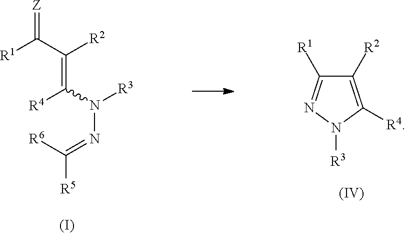

13. A process for the manufacture of a compound of formula (V), which comprises the process according to claim 12, 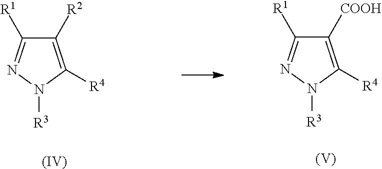
wherein R¹, R², R³ and R⁴ are defined as in claim 12, and further comprises at least one of the steps of
a) when R² is C(O)R¹⁰ and R¹⁰ is selected from the group consisting of CX₃, C₂X₅, n-C₃X₇ or iso-C₃X₇, n-, iso- or tert-C₄X₉, wherein in all of the foregoing groups X is the same or different, selected from the group consisting of F, Cl, Br and I, the compound of formula (IV) is contacted with an aqueous base,
b) when R² is C(O)R¹⁰ and R¹⁰ is selected from the group consisting of C₁-C₁₂-alkyl, optionally substituted C₃-C₁₀-cycloalkyl, optionally substituted aryl, optionally heteroaryl, optionally substituted aralkyl or optionally substituted aralkyl group, the compound of formula (IV) is contacted with an oxidation agent,
c) when R² is CN or C(O)OR⁹, the compound of formula (IV) is contacted with an acid or a base.

14. A process for the manufacture of a compound of formula (VI), which comprises the process according to claim 13, and which further comprises a first step, wherein the compound of formula (V) is reacted with a halogenating agent, an acylating agent or CDI, and a second step, wherein the product from the first step is contacted with a compound of formula (VII), NHR¹⁷Q, wherein R¹⁷ is selected from the group consisting of H, C₁-C₁₂-alkyl, C₂-C₆ alkenyl or C₃-C₈-cycloalkyl group, and wherein Q is an optionally substituted aryl or heteroaryl group.

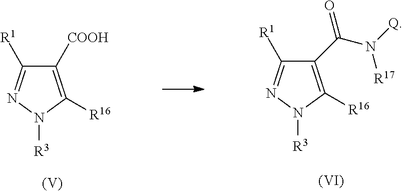

15. A process for the manufacture of a compound of formula (VI), which comprises the process of claim 12, and which further comprises one of the steps d) and e), wherein
d) when R² in the compound of formula (IV) is C(O)R¹⁰ and R¹⁰ is selected from the group consisting selected from the group consisting of CX₃, C₂X₅, n-C₃X₇ or iso-C₃X₇, n-, iso- or tert-C₄X₉, wherein in all of the foregoing groups X is the same or different, selected from the group consisting of F, Cl, Br and I, the compound of formula (IV) is contacted with a compound of formula NHR¹⁷Q, wherein R¹⁷ is selected from the group consisting of H, C₁-C₁₂-alkyl, C₂-C₆ alkenyl or C₃-C₈-cycloalkyl group, and wherein Q is an optionally substituted aryl or heteroaryl group, or
e) when R² in the compound of formula (IV) is C(O)OR⁹, wherein R⁹ is defined as in claim 1, the compound of formula (IV) is contacted with a compound of formula NHR¹⁷Q, wherein R¹⁷ is selected from the group consisting of H, C₁-C₁₂-alkyl, C₂-C₆ alkenyl or C₃-C₈-cycloalkyl group, and wherein Q is an optionally substituted aryl or heteroaryl group, and at least one compound selected of the group consisting of a Lewis acid or a base.

16. The process according to claim 3, wherein the amine salt is derived from a tertiary aromatic amine.

17. The process according to claim 4, wherein the tertiary aromatic amine salt is pyridinium hydrochloride.

18. The process according to claim 14, wherein R¹⁷ is selected from the group consisting of H and C₁-C₄-alkyl.
